(19) United States
(12) Patent Application Publication
(10) Pub. No.: US 2007/0142322 A1
(43) Pub. Date: Jun. 21, 2007

(54) Method of using chitosan to reduce shedding of E. coli from bovines

(76) Inventors: Charles W. Kaspar, Madison, WI (US); Kwang-Cheol Jeong, Madison, WI (US)

Correspondence Address: DEWITT ROSS & STEVENS S.C.;WISCONSIN ALUMNI RESEARCH FOUNDATION, 8000 EXCELSIOR DRIVE, # 401, MADISON, WI, 53717, US

(21) Appl. No.: 11/312,906

(22) Filed: Dec. 20, 2005

(51) Int. Cl. A61K 31/722

(52) U.S. Cl. 514/55

(57) ABSTRACT

A method to reduce fecal shedding of E. coli from bovines is described. The method includes administering an amount of chitosan to a bovine. The amount administered is sufficient to reduce or eliminate fecal shedding of E. coli from the bovine. Also described is a corresponding feed ration for bovines. The feed ration includes chitosan as an active agent to reduce or inhibit shedding of E. coli from bovines, including the shedding of pathogenic E. coli such as strain O157:H7.

### GOVERNMENT SUPPORT

This invention was made with United States government support awarded by the following agency: USDA/CSREES 04-CRHF-0-6055. The United States has certain rights in this invention.

### FIELD OF THE INVENTION

The invention is directed to a method of using chitosan as the active agent in an orally-administered pharmaceutical composition to inhibit the shedding of E. coli from bovines in general and beef cattle in particular.

### BACKGROUND

Zoonotic diseases are diseases caused by infectious agents that can be transmitted between (or are shared by) animals and humans. Escherichia coli (E. coli) infection is one such zoonotic disease. A specific and serious public health concern is infection by E. coil O157:H7. The O157:H7 strain is distinguished microbiologically from other E. coli strains by its inability to ferment sorbitol and, most importantly from the perspective of human health, by its production of “shiga-like” toxins (SLT-I and SLT-II). (SLTs were so named because of their similarity to the toxin of Shigella.)

In humans, infection with the O157:H7 strain of E. coli can be fatal. The organism causes watery diarrhea, hemorrhagic coli tis, and hemolytic-uremia syndrome in humans. It is the hemolytic-uremia syndrome that can result in a fatal outcome. Hemolytic-uremia syndrome is characterized by hemolytic anemia, thrombocytopenia, and ultimately renal failure. It most commonly occurs following infection in children. Approximately 2 to 7% of E. coli O157:H7-infected children develop hemolytic-uremia syndrome. In humans, the shiga-like toxins produced by the E. coli damage vascular endothelium, leading to thrombotic lesions and disseminated intravascular coagulation.

The hemolytic-uremia syndrome probably reflects the same basic process, with thrombotic lesions in the kidneys.

It is important to bear in mind that O157:H7 is just one of a great many serotypes of E. coli that can produce these toxins and cause disease. Moreover, other virulence factors (e.g., intimin, adhesin, and hemolysin) may also be involved in E. coil pathogenesis. From a public health standpoint, however, E. coli O157:H7 is the most important enterohemorrhagic serotype associated with human disease in the United States. Other serotypes of E. coli are also emerging as important pathogens in the U.S. and throughout the world. Among them are E. coli of serotypes O26:H11 and O111:NM which are recognized pathogens for both humans and young calves.

Moreover, enterohemorrhagic E. coli are not the only types of E. coli that cause illness in humans or other mammals. Pathogenic strains of E. coli are also responsible for urinary tract infections and neonatal meningitis in humans. Pathogenic strains of E. coli are characterized using a broad array of virulence determinants, including (by way of a non-limiting list): adhesins (e.g., CFAI/CFAII, Type 1 fimbriae, P fimbriae, S fimbriae, and non-fimbrial adhesin); invasins (e.g., hemolysisn and siderophores); motility/chemotaxis; toxins (e.g., LT toxin, ST toxin, SLT, cytotoxins, endotoxin LPS); antiphagocytic surface properties (e.g., capsules, K antigens, LPS); defense against serum bactericidal reactions; defense against immune responses; and various genetic attributes. (See, for example, Todar's Online Textbook of Bacteriology, compiled and edited by Kenneth Todar of the University of Wisconsin-Madison Department of Bacteriology.) Thus, while a relatively small number of the over 700 antigenic types of E. coli now known are pathogenic, serotyping these strains remains important to distinguish the various pathogenic strains.

Cattle are a principal reservoir of enterohemorrhagic Escherichia coli. Prevalence estimates vary, but it appears that although a substantial percentage of both dairy herds and beef feedlots have infected animals, the actual number of individual infected animals at any one time is relatively low. Recent surveillance data indicate that prevalence rates of E. coli O157:H7 in cattle are much higher than was estimated several years ago. Results of a recent study of cattle at slaughter houses in the U.S. during the summer months revealed E. coli O157:H7 in fecal samples of 28% of animals tested. This high rate of E. coli O157:H7 carriage by cattle substantiates the need for intervention strategies at the point of production to prevent contamination of food and water supplies. See, for example, Doyle, T. Zhao, & P. Zhoa, “Control of EHEC in Cattle by Probiotic Bacteria,” FDA Grant FD-U-00159701 (Sep. 30, 1998-Dec. 31, 2000).

A study of U.S. dairy herds in 2002 (funded by the U.S. Department of Agriculture, National Animal Health Monitoring System) found that 38.5% of dairy farms had at least one cow that was positive for E. coli O157:H7 when sampled, but only 4.3% of individual cows were actively shedding the organism. Infection with O157:H7 is, however, sub-clinical in cattle. The duration of fecal shedding of E. coli from cattle is quite variable and intermittent. Dairy calves and heifers appear to shed the organism more often than adults. The peak time of infection is thought to be 3-18 months of age. Recovery of O157:H7 from beef feedlots is highest in pens from which the cattle had been in the feedlot for the shortest periods of time, suggesting that stress (from handling) plays a prominent role in shedding of the organism. This variability in the timing of fecal shedding makes fecal testing for the organism a far-less-than-ideal tool to predict, manage, or control transmission of the organism from herd-to-herd or from herd-to-human.

It has been reported that several strains of probiotic E. coli can inhibit the growth of E. coli O157:H7 in vitro and reduce or eliminate fecal shedding of E. coli O157:H7 in weaned calves. (See the USDA grant report referenced supra.) At present, however, there is no treatment that predictably and reliably inhibits the fecal shedding of E. coli from bovines in general and beef/dairy cattle in particular.

The need to address the issue is one of primary concern for the public health. E. coli O157:H7 transmission to humans is via the fecal-oral route, and is often associated with eating improperly cooked or prepared animal products (most notably ground beef, but also unpasteurized milk and processed meats). Contaminated vegetable products have also been sources for human outbreaks. E. coli O157:H7 remains viable for more than two months in feces and soil. It survives quite well in ground beef It remains infectious for weeks to months in acidic foods such as mayonnaise, sausage, apple cider and cheddar cheese at refrigeration temperatures. (See, for example, the January 2004 update on E. coli infections from the Center for Food Security and Public Health at the College of Veterinary Medicine at Iowa State University.) Reducing the instances of E. coli infection in humans by reducing the shedding of E. coli from cattle remains a long-felt and unmet need in both the United States and across the globe.

Chitosan is a polysaccharide comprised of repeating glucosamine units. It is produced by the deacetylation (partial or complete) of chitin obtained from the shells of marine arthropods such as shrimp. The patent literature contains a number of patents that reference using chitin in a host of various end-uses. For example, U.S. Pat. No. 6,352,727 describes a topical antibacterial composition containing eucalyptus extract and chitosan. See also U.S. Pat. No. 6,630,458 (which describes a topical composition for treating mastitis in cows). Published U.S. Patent Application 2003 0 129 295 describes a method for protecting hygroscopic materials that uses chitosan as a release agent or density modifier. Published U.S. Patent Application 2005 0 074 440 describes a strain of the probiotic Lactobacillus rhamnosus. The probiotic is administered using chitosan as an excipient. Chitosan has also been touted as a fat replacement for diet foods. See, for example, U.S. Pat. No. 5,718,969.

### SUMMARY OF THE INVENTION

The invention is a method to reduce fecal shedding of E. coli from bovines. The method comprises administering an amount of chitosan or a chitosan derivative to a bovine. The amount of chitosan or chitosan derivative administered is sufficient to reduce or eliminate fecal shedding of E. coli from the bovine.

In the preferred embodiment of the invention, the chitosan or chitosan derivative is administered to the bovine (preferably cattle, although it can be administered to any bovine) in an amount sufficient to reduce or eliminate the fecal shedding of pathogenic E. coli in general, enterohemorrhagic E. coli more specifically, and pathogenic O157:H7, O26:H11, or O111:NM E. coli specifically, from the bovine.

It is preferred that the chitosan or chitosan derivative is administered to the bovine by mouth. The chitosan or chitosan derivative may, however, be delivered directly to the rumen or to the intestine of the bovine by any means now known or developed in the future.

The amount of chitosan or chitosan derivative administered preferably ranges from about 1 g/day to about 1,000 g/day, per bovine, administered in one or more discrete doses. The amount of chitosan administered may also range from about 5 g/day to about 500 g/day, or from about 5 g/day to about 100 g/day, or from about 5 g/day to about 50 g/day per bovine, administered in one or more discrete doses. Ranges above or below these stated ranges are within the scope of the invention claimed herein. The amount of chitosan administered to any given animal on a per day basis is ultimately at the discretion of a veterinarian or husbandryman, taking into account the age, size, sex, bacterial load, and general condition of the bovine to be treated.

It is generally preferred, although not required that the chitosan or chitosan derivative is administered to the bovine in combination with a base feed ration suitable for bovines.

Another embodiment of the invention is a feed ration for bovines that inhibits the fecal shedding of E. coli. The feed ration comprises a base feed ration in combination with an amount of chitosan or chitosan derivative, wherein the amount of chitosan or chitosan derivative present in the feed ration is sufficient to reduce or eliminate fecal shedding of E. coli from bovines fed the feed ration. The amount of chitosan or chitosan derivative present in the feed ration preferably ranges from about 5 g to about 100 g per daily ration per bovine, more preferably from about 5 g to about 500 g per daily ration per bovine, and most preferably from about 1 g to about 1,000 g per daily ration per bovine.

The primary benefit of the invention is that it reduces or eliminates the shedding of E. coli in general, and pathogenic E. coli in particular, from animals destined for human consumption. The invention thereby reduces the potential for E. coli contamination of raw meat products, such as ground beef, and processed meat products, such as cold-cuts, sausage, and the like.

Another benefit of the invention is that it reduces direct E. coli transmission from live animals to human workers at livestock farms, feed lots, finishing lots, livestock holding facilities, slaughterhouse, and the like. By reducing the shedding of E. coli when the livestock is still living, the invention also greatly reduces the potential for downstream E. coli contamination in meat-processing and meat-packing plants.

Another benefit of the invention is that the active ingredient, chitosan, is a commodity product that is widely available in the commercial markets.

### BRIEF DESCRIPTION OF THE DRAWINGS

FIG. 1 is a graph depicting a comparison of E. coli O157:H7 shedding in inoculated cattle receiving low-titer egg antibody or control eggs.

FIG. 2 is a graph depicting a comparison of E. coli O157:H7 shedding in inoculated cattle receiving medium-titer egg antibody or control eggs.

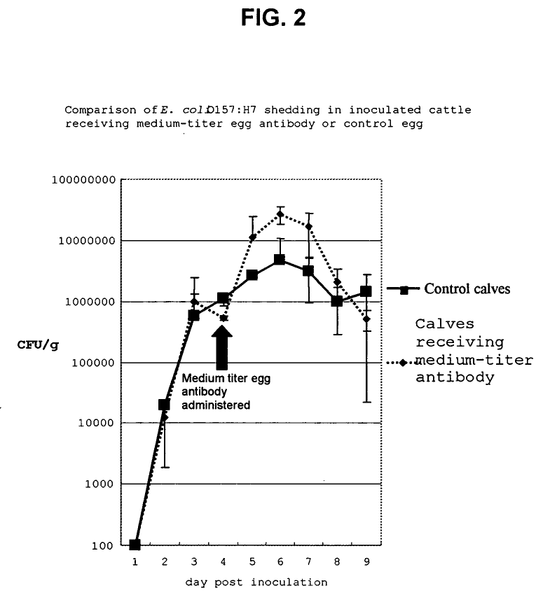

FIG. 3 is a graph depicting a comparison of E. coli O157:H7 shedding in inoculated cattle receiving high-titer egg antibody or control eggs.

FIG. 4 is a graph depicting a comparison of E. coli O157:H7 levels in fecal samples (♦) and swab samples from the rectal-anal junction (▪) in cattle inoculated with E. coli O157:H7 and then fed a diet including chitosan and anti-E. coli O157:H7 antibodies.

FIG. 5 is a graph depicting a comparison of E. coli O157:H7 levels in fecal samples (♦) and swab samples from the rectal-anal junction (▪) in a steer inoculated with E. coli O157:H7 and then fed a diet including chitosan.

FIG. 6 is a graph depicting another comparison of E. coli O157:H7 levels in fecal samples (♦) and swab samples from the rectal-anal junction (▪) in a steer inoculated with E. coli O157:H7 and then fed a diet including chitosan.

### DETAILED DESCRIPTION

As used herein, the term Escherichia coli (E. coli) refers to all E. coli, now known or identified in the future. As used herein the term E. coli explicitly encompasses all known pathogenic strains and sub-strains of E. coli, and all such strains identified in the future. As used herein the term E. coli explicitly encompasses E. coli strains O157:H7, O26:H11, and O111:NM.

As used herein, the term “base feed ration” refers to a nutritionally sufficient base feed ration for bovines such as beef cattle, dairy cattle, buffalo, and the like. A host of suitable base feed rations are well known in the art and can be purchased commercially at any feed mill located in any agricultural area of the United States where beef or dairy cattle are raised and bred. The base feed ration may be a minimal ration comprising only the essential ingredients required to sustain the animal. Or the base feed ration may comprise a total mixed ration containing a host of additional ingredients to render the animal productive for its intended use (e.g., as a dairy cow or as a meat animal). A total mixed ration generally comprises a complete ration which provides the required level of nutrients (ie., caloric content, fiber, protein, minerals and vitamins) needed by the animal to produce, for example, a desired weight of milk, or a desired weight gain. Base feed rations for bovines are well known in the art and will not be described in any greater detail herein.

A large number of test kits for detecting E. coli can be obtained from numerous commercial sources and are well known in the art. Any of the following kits can be used (following the manufacturer's instructions) to detect E. coli shed by bovines, or E. coli from sample swabs taken from bovines. By way of a non-limiting list, the following kits can be purchased in the United States and elsewhere: Coliscan®-brand Easygel® kit (in both an unincubated version and an incubated version), and Coliscan®-brand MF Method kit, distributed by Micrology Laboratories (Goshen, Ind.); 3M Petrifilm®-brand kit, distributed by 3M (St. Paul, Minn.); and the Colisure-brand kit, distributed by IDEXX Laboratories (Westbrooke, Me.).

Similarly, a large number of test kits for specifically detecting O157-positive E. coli can be obtained from numerous commercial sources and are well known in the art. By way of a non-limiting list, the following kits can be purchased in the United States and elsewhere: Tecra-brand E. coli O157 Visual Immunoassay and Tecra-brand E. coli O157 Immunocapture kit (Tecra International Pty Ltd. (Frenchs Forest, Australia); Pathatrix-brand E.coli O157 Test System (Matrix MicroScience Inc., Golden Colo.); RapidCheck-brand test for E. coli O157 (Strategic Diagnostics Inc., Newark, Del.); Pathigen-brand E. coli O157 test kit (formerly sold by Igen International, acquired in July 2003 by Roche Molecular Systems, Inc., Alameda, Calif.); BAX®-brand System PCR Assay for Screening E. coli O157:H7 (Dupont Qualicon, Inc., Wilmington, Del.); Transia Card-brand E. coli O157 test kit (Diffchamb AB, Gothenburg, Sweden); Singlepath-brand E. coli O157 test kit (Merck KGaA, Darmstadt, Germany); Warnex-brand Rapid Pathogen Detection System for E. coli O157 (Warnex Diagnostics, Inc., Laval, Quebec, Canada); Marshfield Clinic E. coli O157:H7 test method (Marshfield Clinic, Marshfield, Wis.); and BBL-brand CHROMagar-brand O157 test kit (BD Diagnostics, Franklin Lakes, N.J.).

Further still, at least one commercially available kit has been purposefully optimized for detecting O157-positive E. coli from fecal samples using real-time polymerase chain reaction technology: the Ruggedized Advanced Pathogen Identification Device (R.A.P.I.D.) System Escherichia coli O157 real-time PCR detection kit (Idaho Technology, Inc., Salt Lake City, Utah).

All of the above-noted kits include detailed manufacturer's instructions on how to use the kit and how to interpret the results of the corresponding tests. Because of the ubiquity and well known characteristics of these commercially available kits, test methods for detecting E. coli from bovines will not be addressed in any greater detail herein.

As used herein the term “chitosan” (CAS 9012-764) refers to chitosan derived from any source, of any degree of deacetylation, of any degree of polymerization, and of any molecular weight. “Chitosan” as used herein explicitly includes chitosan oligomers. Chitosan is available commercially from a host of different sources, in the form of solutions, flakes, fine powders, beads, and fibers, any and all of which can be used in the present invention. Microparticles of chitosan, however, are preferred. Microparticulate chitosan can be obtained, for example, from Alfa Chem (Kings Port, N.Y.), CarboMer, Inc. (San Diego, Calif.), and many others. Methods to fabricate nanoparticulate chitosan are also described in the art. See, for example, Nie et al (2006) Nanotechnology 17:140-144 (published on-line on 1 Dec. 2005), and Qi et al. (2004) Carbohydrate Research 339:2693-2700.

The term “chitosan derivative” refers to a chitosan molecule (as defined in the immediately preceding paragraph) having one or more substituents attached to the chitosan polymer chain. Specifically encompassed within the term “chitosan derivative” are N-substituted chitosan derivates, such as N-alkyl-, N-alkenyl-, and N-alkynyl-substituted chitosan; N-carboxy-substituted chitosan; N-carboxyalkyl-substituted chitosan (e.g., N-carboxymethyl to N-carboxyhexyl derivations of chitosan), and the like.

As noted in the Background section, a spectrum of illnesses are caused by E. coli O157:H7 in humans, ranging from mild diarrhea or hemorrhagic colitis to life-threatening hemolytic uremic syndrome (HUS) and thrombotic thrombocytopenic purpura. Since first being recognized as a food-borne pathogen in 1982, E. coli O157:H7 has emerged as a major food-borne pathogen; epidemiological data indicate that outbreaks of both hemorrhagic colitis and HUS are increasing in number and geographic scope (Griffin, P. M., in “Infections of the Gastrointestinal Tract,” p. 739-761, ©1995 Raven Press, Ltd., New York). Ground beef is most frequently implicated in food-borne outbreaks of E. coli O157:H7. (See, for example, Padhye & Doyle (1992) J. Food Prot. 55:555-565.)

In addition to contaminated foods, E. coli O157:H7 is also transmitted by contaminated water, from person-to-person, and from animal-to-person. These latter modes of transmission suggest that E. coli O157:H7 has a very low infectious dose. Epidemiological data and surveys indicate that cattle are a reservoir (Garber et al. (1995) J Am. Vet. Med. Assoc. 207:46-49), with 7% to 16% of the herds positive for E. coli O157:H7 (Faith et al. (1995) Appl. Environ. MicrobioL 62:1519-1525). Recent data on the prevalence of E. coli O157:H7 (H7 and non-motile) in the feces and on hides of cattle in feedlots was 28% and 11%, respectively (Elder et al. (2000) Proc. Natl. Acad. Sci. 97:2999-3003). In a survey of Wisconsin dairy herds, Faith et al. (1995) found 5 of 70 herds (herd prevalence 7.1%) and 10 of 560 weaned calves (animal prevalence 1.8%) tested positive for E. coli O157:H7. The differences in prevalence between studies are due to the different ages in the animals examined, the various modes of fecal sample collection and handling, the size of sample tested, and varying detection methods.

Another contributing factor to the variation in prevalence between studies is the sporadic nature of E. coli O157:H7 shedding in cattle. (See, for example, Hancock et al. (1997) Epidemiol. Infect. 118:193-195.) Intermittent shedding has been attributed to diet changes, the lack of sensitivity in sampling and detection methods, and the low numbers of E. coli O157:H7 found in feces. E. coli is a minor inhabitant of most gastrointestinal environments and this is true for E. coli O57:H7 when present in cattle. Shere et al. (1998) Appl Environ. Microbiol. 64:1390-1399 found 10² to 10⁴ E. coli O157:H7 CFU per gram of feces in naturally infected cattle. Cattle shed the organism for varying periods of time ranging from 1 to 16 weeks (Besser et al. (1997) J. Infect. Dis. 175:726-729). These results are consistent with inoculation studies that observed shedding in steers from 7 to 14 weeks (Brown et al. (1997) Appl. Environ. Microbiol. 63:27-32).

These findings raise the question of whether the intestinal tract of cattle is “colonized” during the carrier state. It is possible that E. coli O157:H7 is simply a transient and passes through the bovine intestinal tract into the environment where it can re-infect other animals rather than colonizing the intestinal epithelium. Additionally, inoculation and natural infection of cattle does not prevent future shedding of the same strain of E. coli O157:H7 and suggests that immunity has little role in preventing or limiting colonization of the intestinal tract by this bacterium (see Shere et al. (1998), supra).

Naturally-infected and inoculated cattle develop serum antibodies to the O157 antigen, but these antibodies do not protect the animal from subsequent shedding. In a study by Hoffman et al. (1997) (abstract V67/VIII, p. 117, VTEC: 3^{rd} International Symposium and Workshop on Shiga Toxin (Verocytotoxin)-Producing E. coli Infections), oral vaccination of calves with 10¹⁰ CFU of a toxin-negative O157:H7 strain resulted in serum antibodies to the O antigens but did not reduce shedding of the parent E. coli O157:H7 strain following inoculation. These investigators also noted that Shiga toxin 2 (Stx2) had an immunosuppressive effect and suggested that antibodies to Stx2 may be needed to supplement antigens or organisms used as vaccines in order to maximize the immune response.

Intimin has received attention as a potential vaccine because of its role in adhesion to intestinal epithelial cells and because a majority of strains implicated in human disease produce intimin. However, cattle shed intimin-negative, Shiga-toxin positive E. coli at a higher frequency than intimin-positive strains (Sanhu et al. (1996) Epidemiol. Infect. 116:1-7.) This raises the question of whether intimin plays any role in E. coli carriage in cattle.

Passive immunity plays an important role in protecting calves from scours caused by enterotoxigenic E. coli expressing the K99 pili. However, this approach has not been used for E. coli O157:H7 and other Shiga toxin-producing E. coli because the amount of antibody necessary to protect adult cattle would be cost prohibitive.

The principal aim of the present invention is to control E. coli O157:H7 in fresh meat products in general, and fresh beef products in particular, by intervening at the pre-harvest level, while the livestock is still living. Conventionally, control practices for E. coli O157:H7 are primarily applied at the processing level. The most common conventional treatments of carcasses include physically removing visible fecal contamination, washing with hot water or acid, and treating with steam. Despite implementing these practices, there continues to be a significant number of recalls and beef-associated illnesses associated with E. coli O157:H7.

The crux of the invention rests on the belief that a further reduction in the prevalence of E. coli O157:H7 in beef requires positive intervention at a different point in the farm-to-consumer continuum. “On-farm” control practices have received little attention, although administering competitive microorganisms and interrupting waterborne transmission within cattle herds have been proposed as possible strategies to reduce the prevalence and duration of shedding within cattle herds.

The present invention, however, can be implemented “on-farm” and/or immediately pre-harvest (at the finishing lots or slaughterhouse holding pens). It is envisioned that the present invention will complement existing processing treatments to control the transmission of pathogenic E. coli in general and E. coli O157:H7 in particular.

At the heart of the invention is the very unexpected discovery that chitosan acts as an active agent to reduce or to eliminate entirely the shedding of E. coli in general (and E. coli O157:H7 specifically) in the feces of bovines. This discovery was the serendipitous result of the experiments described in Examples 1 through 4, described below. Thus, the invention is a method to reduce fecal shedding of E. coli from bovines, the method comprising administering an amount of chitosan to a bovine, wherein the amount is sufficient to reduce or eliminate fecal shedding of E. coli from the bovine. The invention also includes a feed ration to inhibit fecal shedding of E. coli. The feed ration comprises a base feed ration in combination with an amount of chitosan, wherein the amount of chitosan present in the feed ration is sufficient to reduce or eliminate fecal shedding of E. coli from bovines fed the feed ration.

### EXAMPLES

The following Examples are included solely to provide a more complete disclosure of the invention described and claimed herein. The Examples do not limit the scope of the invention in any fashion.

### Example 1

### Initial Feeding Trial Using Anti-E. coli O157:H7 Antibodies

Laying hens were immunized with formalin-fixed cells of E. coli O157:H7 strain 86-24. Eggs were collected from each bird prior to immunization to compare titers of anti-O157 antibodies before and after immunization. Eggs were collected from immunized birds starting one week after the last injection. The titers of anti-O157 immunoglobulins from pre-immunization eggs were <32 and increased significantly following immunization (ranging from 256 to 1024). The average weight of egg yolks was approximately 7 grams and the antibody concentration ranged from about. 1.5 to 6 mg per ml (gram). Thus, the quantity of antibody per egg varied from about 10 to about 40 mg per egg. Cattle shedding E. coli O157:H7 were fed control eggs (either whole raw egg or freeze dried) or eggs containing anti-O157 antibody either as whole raw egg (5 eggs per day, approximately 100 mg of anti-O157 antibody), or as freeze-dried powder (30 grams of dry egg which constitutes about 375 mg of anti-O157 antibody). The eggs were fed to cattle starting on day 4 post-inoculation.

The average number of E. coli O157:H7 shed by cattle fed control eggs vs. eggs containing anti-O157 antibodies were essentially the same. The significance of this Example is that feeding any given type of anti-E. coli O157:H7 antibody to bovines will not necessarily inhibit the shedding of E. coli from the bovines.

### Example 2

### Second Feeding Trial Using a Different Anti-E. coli O157:H7 Antibody

Based upon the results from Example 1, the immunogen used to immunize chickens was changed. In this Example, four (4) E coli O157:H7 strains isolated from cattle were grown in M10 medium (minimal medium based upon intestinal contents) anaerobically. These cells were harvested by centrifugation and lysed using B-PER-brand bacterial protein extraction kit (Pierce Biotechnology, Rockford, Ill.). The insoluble fractions of these extracts, which are partly comprised of outer membrane proteins (OMP) from the E. coli, were pooled and used as an immunogen to inoculate laying hens. A portion of the extracts from each strain was retained and used for protein comparisons by polyacrylamide gel electrophoresis for comparisons of aerobically and anaerobically grown E. coli O157:H7. Anaerobically grown cells contained three proteins not evident in aerobically grown cells and another three proteins that were present at greater quantities. These extracts produced titers of anti-O157 antibodies in eggs ranging from about 25 to about 50 mg per egg. Based upon the titers ofanti-O157 antibody in eggs, the eggs from chickens were split into high, medium, or low categories. Eggs in each category were pooled and freeze-dried. Cattle were randomly assigned to receive control eggs or one of the categories of eggs containing anti-O157 antibodies at 40 g per day starting four days after inoculation and continuing to day 9. It is estimated that cattle received daily approximately 500, 300, and 100 mg of anti-O157 egg antibody in the high, medium, and low categories, respectively.

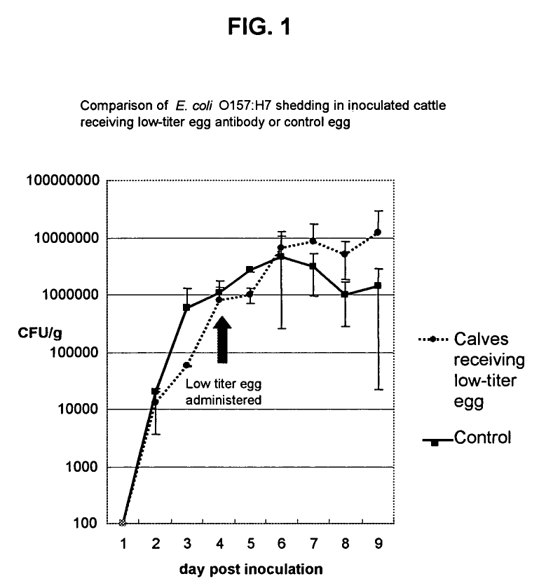

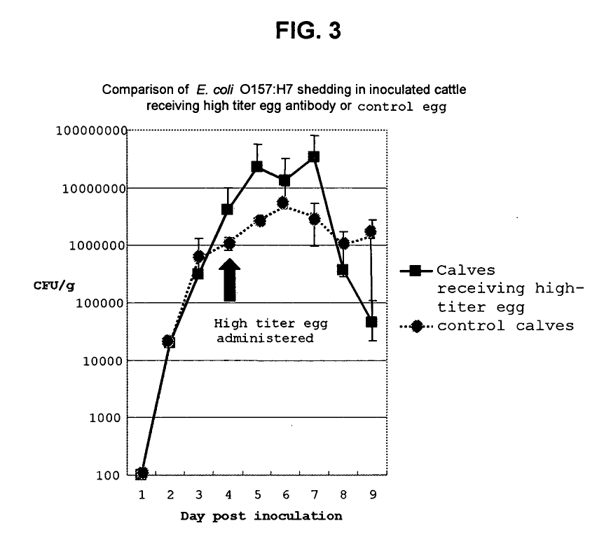

Results from this study are shown in FIGS. 1 (low titer), 2 (medium titer), and 3 (high-titer). The average number of E. coli O157:H7 shed by cattle receiving the same category of egg antibody and control animals is presented. There was not a significant difference in the number of E. coli O157:H7 shed between the control cattle and antibody-fed cattle, even in cattle receiving eggs with the highest titer of anti-O157 antibodies (see FIG. 3). However, there appeared to be a trend, or impact, when comparing data from cattle receiving low- to high-titer eggs, namely a lag of 6 to 7 days prior to seeing a drop in the amount of E. coli shed. The lag before a drop in the amount of shed E. coli was expected considering that the transit time through the ruminant intestinal tract is 2 to 3 days (depending upon the diet). Thus, the results suggest that with the use of concentrated or protected antibodies to specific bacterial surface targets, a reduction in the numbers of E. coli O57:H7 shed by cattle can be accomplished.

### Example 3

### Generation of Antibodies to Targeted E. coli Antigens

The results from Examples 1 and 2 indicate that the immunogen used to generate egg antibodies was an important aspect to the success of passive immunity to reduce the number of E. coli O157:H7 shed in cattle. Accordingly, this Example focused on the production of antibodies to defined targets, such as the Tir receptor (the receptor for intimin) and the outer membrane proteins of E. coli O157:H7. Additionally, antibody was isolated from eggs by polyethylene glycol precipitation in order to administer high-titer, standardized concentrations of antibody to the cattle.

Secreted proteins from E. coli O157:H7 strain ATCC 43895 (American Type Culture Collection, Manassas, Va.) were used as an immunogen to inoculate laying hens. The choice of immunogen was based upon previously described attachment factors, such as Tir and other secreted proteins that are known to stimulate antibody production in infected humans. The methods employed to immunize laying hens and to concentrate antibody worked exceptionally well, with over 40 g of antibody to E. coli O157:H7-secreted proteins recovered. An enzyme-linked immunosorbent assay (ELISA) was used to quantitate antibody from egg fractions and Western blot analysis confirmed that the antibodies reacted with secreted proteins from E. coli O157:H7. Previous results indicated that high-titer antibody preparations (>1 g per day) were necessary to impact the numbers of E. coli O157:H7 shed likely due to microbial degradation of antibody in the rumen. Therefore, the antibody was incorporated into chitosan microparticles.

### Example 4

### Chitosan as an Antibody Carrier for Rumen Bypass

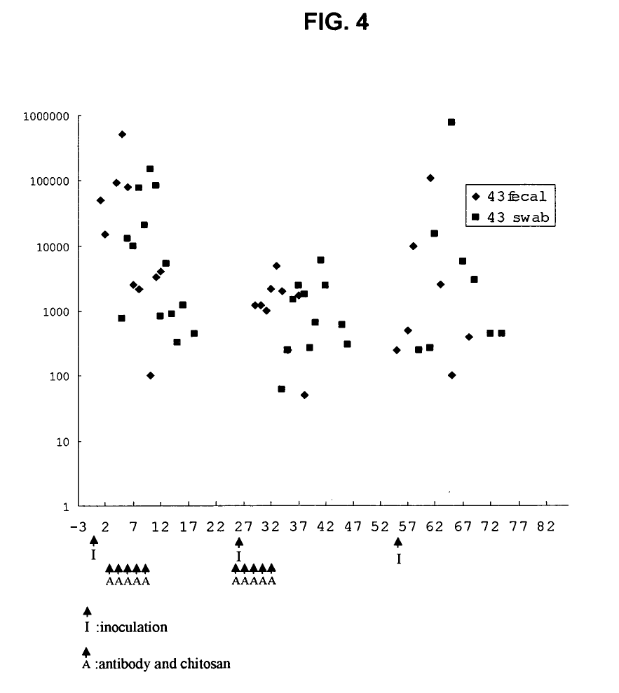

Antibodies to secreted O157 proteins were administered in chitosan microparticles to inoculated cattle at 2 g per day for 5 days while control cattle received chitosan (no antibodies) or no antibody or chitosan (positive control). (The inoculated cattle were previously dosed with approximately 1 million cells of E. coli O157:H7.) The feeding of antibodies in chitosan microparticles or chitosan alone resulted in a reduction of 3 to 4 log₁₀ CFU/g during the period of antibody administration. However, the shedding in control cattle also decreased during this same time frame. The animal-to-animal variation in shedding made it impossible to conduct any meaningful statistical analysis of the data. This variation was overcome by retaining the persistently shedding animals for repeated inoculation and testing. This modification enabled the impact of antibody/chitosan feeding to be assessed. The results are shown in FIGS. 4, 5, and 6.

FIG. 4 contains data from animal 43 that was used in three consecutive trial periods. The E. coli O157:H7 were allowed to clear from the animal (the animal tested negative on four or more consecutive fecal/swab samples) before the start of the next trial. Animal 43 received antibody-chitosan (A) following inoculation (I) in trials one and two. The administration of antibody-chitosan appeared to cause some reduction in fecal shedding when compared to other untreated animals (control) and when compared to trial 3 conducted with this animal. However, the average number shed and the average duration of shedding from trials 1 and 2 did not significantly differ from trial 3. The feeding of the antibody microparticles prior to inoculation appeared to depress the level of E. coli O157:H7 shed (difference between trials 1 and 2). The antibody microparticles appeared to reduce the numbers and duration of shedding of E. coli O157:H7 in the feces to a greater extent than cells associated with the rectal-anal junction (comparison of fecal and swab samples). These results also indicate that the prior inoculation of cow 43 did not protect or significantly alter shedding of the O157:H7 inoculation strain.

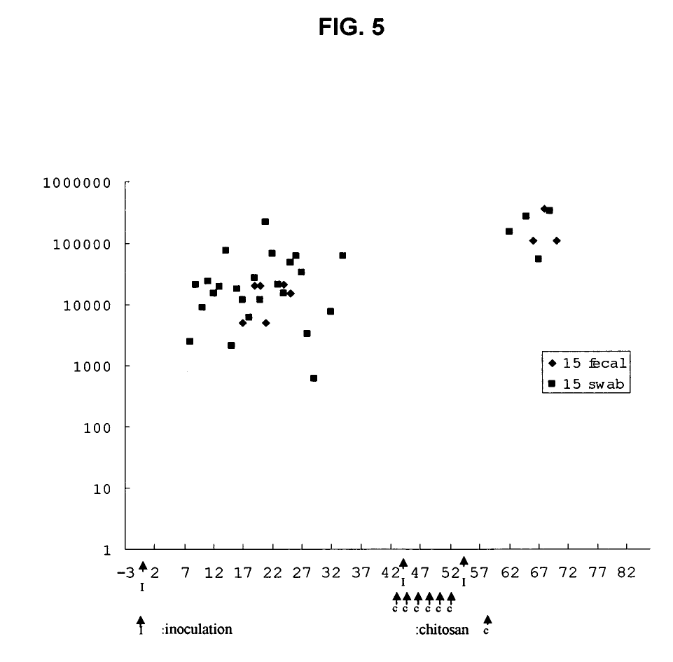

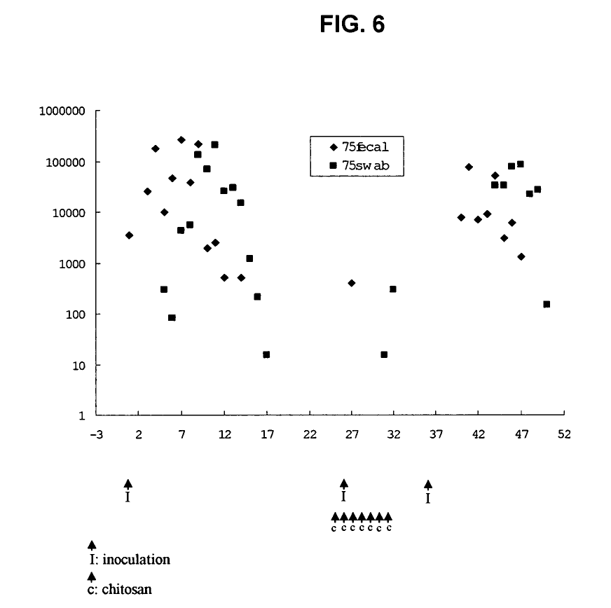

FIGS. 5 and 6 present data from two different animals that were both inoculated without treatment (i.e., without being fed chitosan; trial 1), inoculated and fed chitosan [c] only (trial 2), and inoculated without treatment (trial 3). The last trial (number 3) was conducted to address the possibility that immunity was the cause for the decreased number and duration of shedding noted in trial 2. In animal 15 (FIG. 5), shedding in trials 1 and 3 was essentially the same although the duration was shorter in trial 3 but the numbers shed were generally higher. The shedding in animal 75 (FIG. 6) in trials 1 and 3 was similar. Again, these findings indicate that immunity did not significantly impact shedding of the strain employed and was not the cause of reduced shedding in trial 2.

The key observation from this Example was that shedding of E. coli O157:H7 was significantly reduced in both of the animals that were administered only chitosan. In fact, no E. coli O157:H7 at all was detected in animal 15 and E. coli O157:H7 was detected in only three samples from animal 75.

The significance of this Example is that chitosan alone was found to be active to reduce the shedding of E. coli from bovines. This result was entirely unexpected and entirely unpredictable.

### Example 5

### Crossover Experiment to Assess the Influence of Chitosan

Based upon the unexpected findings made with feeding chitosan alone, an experiment was designed (with thanks to Peter Crump, a statistician at the University of Wisconsin-Madison) to address whether feeding chitosan microparticles alone as the active ingredient causes a statistically significant reduction in fecal shedding of E. coli O157:H7 in bovines. A crossover design was employed to account for the natural animal-to-animal variation in shedding of this organism. The results from this experiment are shown in Table 1 below.

TABLE 1
Crossover design for evaluating the influence of chitosan feeding on the
shedding of Escherichia coli O 157:H7 in inoculated Holstein bull calves

| | No. of positive samples during the 14 days following | | | | | | | |
|---|---|---|---|---|---|---|---|---|
| | inoculation/number of samples | | | | | | | |
| | CONTROL | | | | CHITOSAN | | | |
| Animal | | | >103/g | | | | >103/g | |
| Number | fecal | swab | or swab | total | fecal | swab | or swab | total |
| 12 | 13/13 | 10/10 | 16/23 | 23/23 | 12/14 | 12/13 | 13/27 | 24/27 |
| 13 | 11/14 | 9/13 | 16/27 | 20/27 | 2/14 | 0/13 | 1/27 | 2/27 |
| 14 | 11/13 | 9/10 | 13/23 | 20/23 | 0/14 | 13/13 | 0/27 | 13/27 |
| 15 | 12/14 | 12/13 | 9/27 | 24/27 | 10/13 | 8/9 | 10/22 | 18/22 |
| 16 | 0/13 | 1/10 | 0/23 | 1/23 | 8/14 | 8/13 | 11/27 | 16/27 |
| 17 | 10/14 | 12/13 | 9/27 | 22/27 | 2/13 | 2/10 | 0/23 | 4/23 |
| 18 | 12/14 | 12/13 | 11/27 | 24/27 | 9/13 | 6/10 | 0/23 | 15/23 |
| 22 | 11/13 | 5/10 | 14/23 | 16/23 | 9/14 | 6/13 | 10/27 | 15/27 |
| Totals | 80/108 | 70/92 | 88/200 | 150/200 | 52/109 | 55/94 | 45/203 | 107/203 |
| % positive | 74 | 76 | 44 | 75 | 48 | 59 | 22 | 53 |

Statistical analysis of the above data showed that animals fed chitosan alone as the active agent had significantly lower numbers of positive samples (total; p<0.05) and lower positive fecal samples (p<0.05) than animals fed the control diet that lacked chitosan. Feeding chitosan did not influence the number of samples containing >10³ CFU/g or the percent of positive swabs of the rectal-anal junction. Without being limited to any particular mechanism, these data suggest that chitosan interacts with E coli O157:H7 in the lumen, rather than cells associated with the rectal-anal junction, at least at the concentrations tested.

An additional note: one animal still shedding at the end of this Example was administered the remainder of the chitosan on hand (20 g/day) for two days. The treatment eliminated detectable E. coli O157:H7 in fecal and swab samples by day 3.

### Significance of the Examples

In the initial tests using chitosan microparticles as a carrier for O157 antibodies, the control animals were observed to have decreased shedding of E. coli O157:H7. This was unexpected and quite surprising. It thus led to a crossover study to address the impact of chitosan feeding on the shedding of E. coli. Chitosan was shown to exert a statistically significant reduction on E. coli O157:H7 shedding.

What is claimed is:

1. A method to reduce fecal shedding of E. coli from bovines, the method comprising administering an amount of chitosan or a chitosan derivative to a bovine, wherein the amount is sufficient to reduce or eliminate fecal shedding of E. coli from the bovine.

2. The method of claim 1, wherein the amount is sufficient to reduce or eliminate fecal shedding of pathogenic E. coli from the bovine.

3. The method of claim 1, wherein the amount is sufficient to reduce or eliminate fecal shedding of enterohemorrhagic E. coli from the bovine.

4. The method of claim 1, wherein the amount is sufficient to reduce or eliminate fecal shedding of pathogenic O157:H7, O26:H11, or O111:NM E. coli from the bovine.

5. The method of claim 1, wherein the chitosan or chitosan derivative is administered to the bovine by mouth.

6. The method of claim 1, wherein the chitosan or chitosan derivative is administered to cattle.

7. The method of claim 1, wherein the chitosan or chitosan derivative is administered to cattle by mouth.

8. The method of claim 1, wherein the amount of chitosan or chitosan derivative administered ranges from about 1 g/day to about 1,000 g/day, per bovine, administered in one or more discrete doses.

9. The method of claim 1, wherein the amount of chitosan or chitosan derivative administered ranges from about 5 g/day to about 500 g/day, per bovine, administered in one or more discrete doses.

10. The method of claim 1, wherein the amount of chitosan or chitosan derivative administered ranges from about 5 g/day to about 100 g/day, per bovine, administered in one or more discrete doses.

11. The method of claim 1, wherein the amount of chitosan or chitosan derivative administered ranges from about 5 g/day to about 50 g/day, per bovine, administered in one or more discrete doses.

12. The method of claim 1, wherein the amount is administered in combination with a base feed ration suitable for bovines.

13. A feed ration for bovines comprising a base feed ration in combination with an amount of chitosan or chitosan derivative, wherein the amount of chitosan or chitosan derivative present in the feed ration is sufficient to reduce or eliminate fecal shedding of E. coli from bovines fed the feed ration.

14. The feed ration of claim 13, wherein the amount of chitosan or chitosan derivative present in the feed ration ranges from about 1 g to about 1,000 g per daily ration per bovine.

15. The feed ration of claim 13, wherein the amount of chitosan or chitosan derivative administered ranges from about 5 g to about 500 g per daily ration per bovine.

16. The feed ration of claim 13, wherein the amount of chitosan or chitosan derivative administered ranges from about 5 g to about 100 g per daily ration per bovine.

17. The feed ration of claim 13, wherein the amount of chitosan or chitosan derivative administered ranges from about 5 g to about 50 g per daily ration per bovine.
